(19) United States
(12) Patent Application Publication
(10) Pub. No.: US 2026/0000829 A1
(43) Pub. Date: Jan. 1, 2026

(54) SYSTEM AND METHOD FOR VENTED NEEDLE HUB FOR INFUSION

(71) Applicant: KORU Medical Systems, Inc., Mahwah, NJ (US)

(72) Inventors: SUMANTH CHAKRAVARTHY, Flemington, NJ (US); ADAM EDELHAUSER, Kinnelon, NJ (US); BRIAN CHRISTOPHER CASE, Lake Villa, IL (US)

(73) Assignee: KORU Medical Systems, Inc., Mahwah, NJ (US)

(21) Appl. No.: 18/760,544

(22) Filed: Jul. 1, 2024

(51) Int. Cl. A61M 5/158; A61M 5/142; A61M 39/22

(52) U.S. Cl. CPC A61M 5/158; A61M 5/142; A61M 39/22; A61M 2205/02

(57) ABSTRACT

Provided is a vented needle hub assembly for infusion of a liquid into subcutaneous tissue. More specifically, the vented needle hub assembly is provided by a needle hub having an inlet side and an outlet side and a fluid passageway therebetween; an infusion needle in fluid contact with the fluid passageway and extending from the outlet side; a flexible tubing joined to the inlet side in fluid contact with the fluid passageway; at least one vent disposed in a sidewall of the needle hub, the vent in fluid contact with the fluid passageway; and a porous plug disposed within each at least one vent adjacent to the fluid passageway, the porous plug structured and arranged to distinguish gas from liquid, gas permitted to pass from the fluid passageway through the porous plug and out the at least one vent. An associated method of use is provided as well.

### FIELD OF THE INVENTION

The present invention relates generally to medical devices, and more specifically to a vented needle, or needle hub, as used for infusion, the vent permitting the escape of air from the infusion line prior to the delivery of liquid into the patient.

### BACKGROUND

Liquid pharmaceuticals are commonly delivered to patients through injection or infusion. With either type of delivery, it is generally preferable to eliminate air from the delivery system. With a syringe and basic injection, the operator is generally skilled and the amount of air to be purged may be very little—some in the syringe as well as the needle, and a simple depression of the plunger easily disgorges the air from both, with the user releasing pressure upon the plunger as soon as liquid is observed.

With infusion systems, the process can be more trying. Air may exist in both the reservoir from which the liquid is provided—often a syringe, but also throughout the lengths of infusion tubing and their associated needles. With subcutaneous infusion systems, it is not uncommon for multiple needles to be disposed into the receiving therapy patient, and as each tubing and needle element is distinct, each has its own initial volume of air or sterile gas.

Typically, an operator will turn on the infusion pump and drive liquid throughout the system until liquid is observed at each needle. But this can be problematic in several ways. With home infusion therapy care, the operator may not be a true medical practitioner, and he or she may not be quick to realize that the system has been purged, thus leading to excess runoff at the needles which may make insertion messy, and potentially difficult for the needles to be temporarily affixed to the skin as it is slick with liquid. In addition, some medications can cause irritation, if not pain, if disposed upon a person's skin. The operator may also be slow to shut off the pump leading to excessive loss of treatment liquid, which of course could lead to a shortage of the intended dose.

With intravenous infusion, the issue of air or gas introduced through a needle can have immediate and life-threatening consequences. Although subcutaneous infusion is as the name infers, not performed by injecting the liquid into a vein or other primary blood circulation structure, the introduction of air or gas into the subcutaneous tissues can be discomforting and undesirable.

Thus, for at least the issues of mitigating loss of treatment liquid, ensuring proper dosage, adhesion of the needles to the surface of the injection site, ease and comfort of the patient, and of course allowing the administrating party to place the needles and instigate the treatment session with ease and their own sense of comfort, the issue of purging air or sterile gas from an infusion system can be stressful to both the patient and the care giver.

Hence there is a need for a vented needle hub to overcome one or more of the above identified challenges.

### SUMMARY OF THE INVENTION

Our invention solves the problems of the prior art by providing novel systems and methods for a vented needle hub for infusion.

In particular, and by way of example only, according to one embodiment of the present invention, provided is a vented needle hub assembly for infusion of a liquid into subcutaneous tissue, including: a needle hub having an inlet side and an outlet side and a fluid passageway therebetween; an infusion needle in fluid contact with the fluid passageway and extending from the outlet side; a flexible tubing joined to the inlet side in fluid contact with the fluid passageway; at least one vent disposed in a sidewall of the needle hub, the vent in fluid contact with the fluid passageway; and a porous plug disposed within each at least one vent adjacent to the fluid passageway, the porous plug structured and arranged to distinguish gas from liquid, gas permitted to pass from the fluid passageway through the porous plug and out the at least one vent.

In yet another embodiment, provided is a vented needle hub assembly for infusion of a liquid into subcutaneous tissue, including: a needle hub having a tubing inlet at a first location and a needle extending from a second location, and at least one sidewall; a fluid passageway between the tubing inlet and the needle; at least one vent in the needle hub between the first location and the second location, the at least one vent in fluid communication with the fluid passageway; and a porous plug disposed within each at least one vent adjacent to the fluid passageway; wherein the porous plug has a first air flow resistance lower than a second air flow resistance of subcutaneous tissue, and a first liquid flow resistance higher than second liquid flow resistance of subcutaneous tissue.

For yet another embodiment, provided is a vented needle hub assembly for infusion of a liquid into subcutaneous tissue, including: a needle hub having a first end and a second end and a fluid passageway therebetween, the first end joined with a flexible tubing line and the second end joined with a needle having a first length; at least one vent in the needle hub between the first location and the second location, the at least one vent in fluid communication with the fluid passageway; and a porous plug disposed within each at least one vent adjacent to the fluid passageway, the porous plug structured and arranged to permitted gas to pass from the fluid passageway through the porous plug and out the at least one vent.

Further still, in yet another embodiment provided is a method of providing a vented needle hub assembly for infusion of a liquid into subcutaneous tissue, including: providing a needle hub having an inlet side and an outlet side and a fluid passageway therebetween; providing an infusion needle in fluid contact with the fluid passageway and extending from the outlet side; providing a flexible tubing joined to the inlet side in fluid contact with the fluid passageway; providing at least one vent disposed in a sidewall of the needle hub, the vent in fluid contact with the fluid passageway; providing a porous plug disposed within each at least one vent adjacent to the fluid passageway, the porous plug structured and arranged to distinguish gas from liquid, gas permitted to pass from the fluid passageway through the porous plug and out the at least one vent.

For still yet another embodiment, provided is a method of providing infusion therapy into subcutaneous tissue with a vented needle hub assembly, including: providing a needle hub having: an inlet side and an outlet side and a fluid passageway therebetween; an infusion needle in fluid contact with the fluid passageway and extending from the outlet side; a flexible tubing joined to the inlet side in fluid contact with the fluid passageway; at least one vent disposed in a sidewall of the needle hub, the vent in fluid contact with the fluid passageway; a porous plug disposed within each at least one vent adjacent to the fluid passageway, the porous plug structured and arranged to distinguish gas from liquid, gas permitted to pass from the fluid passageway through the porous plug and out the at least one vent; coupling the flexible tubing to a liquid reservoir, the flexible tubing containing a quantity of air; disposing the needle into the subcutaneous tissue of a patient; dispensing liquid from the reservoir into the flexible tubing, the liquid propagating the air towards the needle hub, the air venting through the porous plug, the liquid reaching the porous plug and continuing down through the needle into the subcutaneous tissues.

### BRIEF DESCRIPTION OF THE DRAWINGS

FIG. 1A is a front perspective view of a vented needle hub assembly having at least one porous plug disposed in a vent in accordance with at least one embodiment of the present invention;

FIG. 1B is a front perspective view of a vented needle hub assembly having a ring of porous plug material disposed upon a plurality of vents in accordance with at least one embodiment of the present invention;

FIG. 1C is a front perspective view of a vented needle hub assembly having one or more porous plugs disposed in sidewalls (including the top) of the needle hub, and/or at the inlet of the needle hub, and/or in the flexible tubing in accordance with at least one embodiment of the present invention

FIG. 2A is a front perspective exploded view of a vented needle hub as shown in FIG. 1A in accordance with at least one embodiment of the present invention;

FIG. 2B is a front perspective exploded view of a vented needle hub as shown in FIG. 1B in accordance with at least one embodiment of the present invention;

FIG. 3 is an enlarged cut through side view of a vented needle hub in accordance with at least one embodiment of the present invention;

FIG. 4 is an enlarged cut through side view of the vented needle hub of FIG. 3, disposed into the subcutaneous tissues of a patient, and demonstrating the porous plug venting air/gas in accordance with at least one embodiment of the present invention;

FIG. 5 is an enlarged cut through side view of the vented needle hub of FIG. 3, disposed into the subcutaneous tissues of a patient, and demonstrating the porous plug having vented the air/gas and now blocking liquid which is delivered into the subcutaneous tissues of the patient in accordance with at least one embodiment of the present invention;

FIG. 6 presents a conceptual illustration of an infusion procedure utilizing at least one vented needle hub in accordance with at least one embodiment of the present invention;

FIG. 7 is a flow diagram of a method for providing a vented needle hub in accordance with at least one embodiment of the present invention; and

FIG. 8 is a flow diagram of a method for providing infusion therapy with at least one vented needle hub in accordance with at least one embodiment of the present invention.

### DETAILED DESCRIPTION

Before proceeding with the detailed description, it is to be appreciated that the present teaching is by way of example only, not by limitation. The concepts herein are not limited to use or application with a specific system or method for a vented needle hub for infusion without pre-priming, whether intravenous or subcutaneous, an infusion kit or other easily foreseeable alternative options or configurations where the venting of air or sterile gas is desired before a medicant or liquid is introduced to a patient. Thus, although the instrumentalities described herein are for the convenience of explanation shown and described with respect to exemplary embodiments, it will be understood and appreciated that the principles herein may be applied equally in other types of systems and methods involving a vented needle hub for infusion.

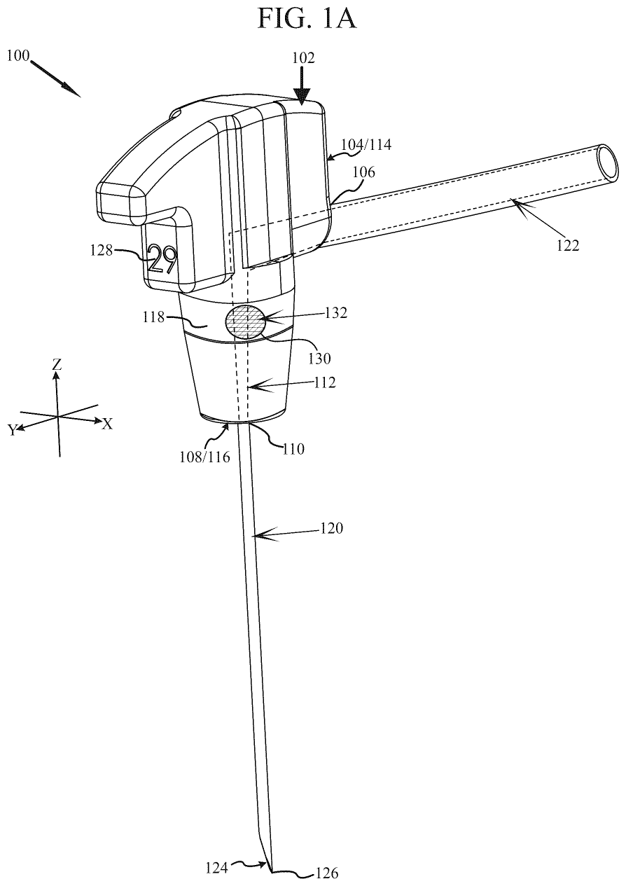

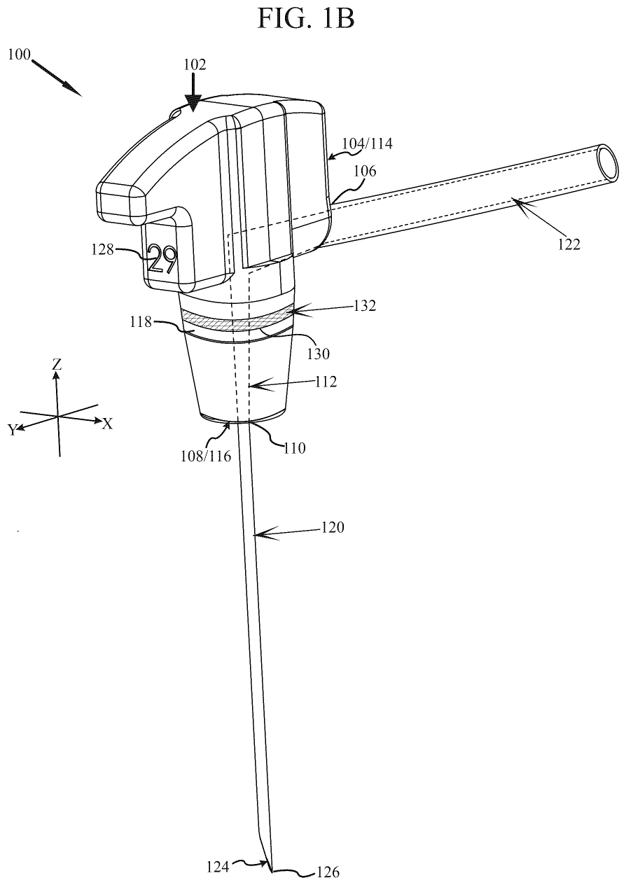

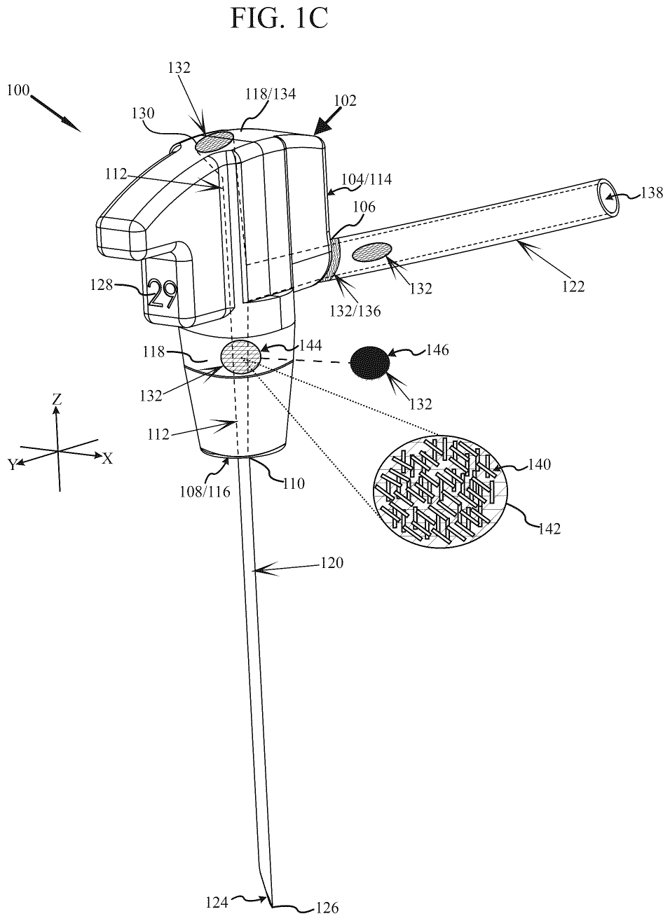

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIGS. 1A and 1B, there is shown at least one vented needle hub assembly 100, hereinafter VNH 100, in accordance with at least one embodiment of the present invention. To facilitate the description of systems and methods for embodiments of VNH 100, the orientation of VNH 100 as presented in the figures is referenced to the coordinate system with three axes orthogonal to one another as shown in FIG. 1A. The axes intersect mutually at the origin of the coordinate system, which is chosen to be the center of VNH 100, however the axes shown in all figures are offset from their actual locations for clarity and ease of illustration.

Continuing with FIG. 1A, presenting a front perspective view, for at least one embodiment, VNH 100 is generally provided by a plurality of elements, the most specific of which is perhaps the needle hub 102, having an inlet side 104, providing an inlet 106, and an outlet side 108 providing an outlet 110, and a fluid passageway 112 therebetween. The inlet side 104 may also be described as a first location 114 providing an inlet 106, and the outlet side 108 as a second location 116 providing an outlet 110.

It will also be appreciated that there is at least one sidewall 118 proximate to the fluid passageway 112 and disposed between the inlet side 104 and the outlet side 108. It will of course be understood and appreciated that the “top” of the needle hub 102 is also a sidewall 118.

An infusion needle 120 is joined to the needle hub 102, and more specifically to the outlet 110 so as to be in fluid contact with the fluid passageway 112. Similarly, flexible tubing 122 is joined to the needle hub 102, and more specifically to the inlet 106 so as to be in fluid contact with the fluid passageway 112.

As will be further discussed and described below, the infusion needle 120 is understood and appreciated to have a first length, and to be selected from a variety of different needle gauge options, such as, but not limited to, 24- to 30-gauge options. This first length will also be understood to be substantially the length of the needle protruding beyond the outlet side 108, to the opening 124 generally proximate to the distal tip 126 from which the liquid is delivered.

For at least one embodiment, the needle hub 102 may also provide indica 128 to indicate the gage and/or length of the infusion needle 120.

As used herein, the term “flexible” as applied to the tubing 122 is understood and appreciated to mean that the tubing 122 is relatively pliable and will easily conform by bending and twisting by an operator. For at least one embodiment, the flexible tubing 122 has a luer connector structured and arranged for connection to a liquid source. Further still, for at least one embodiment the flexible tubing 122 is flow control tubing which may also be referred to as flow rate control tubing.

As shown in FIG. 1A, for at least one embodiment, the needle hub 102 receives the flexible tubing 122 and the infusion needle 120 in a configuration such that they are substantially normal to each other, such that when the needle is disposed down into subcutaneous tissue, the flexible tubing is generally parallel to the patient's skin. Moreover, for at least one embodiment it will therefore be appreciated that there is essentially a 90° transition between the flexible tubing 122 and the infusion needle 120.

In other words, for at least one embodiment the inlet 106 is normal/perpendicular to the outlet 110. It will be understood and appreciated that such a normal/perpendicular relationship between the flexible tubing 122 and the infusion needle 120 is not required for all embodiments of the present invention. Indeed, for yet another embodiment, the inlet 106 and the outlet 110 are substantially opposite or parallel to each other, such that the flexible tubing 122 and the infusion needle 120 are essentially parallel as well.

There is also at least one vent 130 disposed in the sidewall 118 of the needle hub 102, the vent in fluid contact with the fluid passageway 112. A porous plug 132 is disposed within each vent 130 adjacent to the fluid passageway 112. As shown in FIG. 1A there is one vent 130 with a porous plug 132. However, it will be appreciated that for yet another embodiment a second vent and plug may be provided on the opposite side-which is not presently visible. As noted above, the top 134 of the needle hub 102 is also a sidewall 118. For at least one embodiment the fluid passageway 112 may be extend to the top 134, such that the porous plug 132 is more easily observed by users, as is shown in FIG. 1C. Moreover, for at least one embodiment the fluid passageway 112 may be described as having a “T” or “Y” shape configuration.

As shown in FIG. 1B, for yet another embodiment, the porous plug 132 is provided as ring 132 of porous material that is disposed about a portion of the needle hub 102 so as to cover one or more vents 130 in fluid contact with the fluid passageway 112.

Returning to FIG. 1C, an additional embodiment is shown wherein the porous plug 132 is provided as an element of the inlet 106, either as one or more plug elements disposed in one or more specific vents, or as an annular ring 136 affixed to the inlet, and to which the flexible tubing 122 is directly or at least partially coupled. For such an embodiment, physical space between the end of the flexible tubing 122 and the needle hub 102 that is defined at least in part by the porous plug 132 is understood and appreciated to be the vent 132 providing gas or liquid from the flexible tubing 122 to contact the porous plug 132/136.

For yet another embodiment, the at least one vent 130 may be established in the flexible tubing 122 such that the porous plug 132 is more properly understood and appreciated to be in fluid contact with the fluid channel 138 within the flexible tubing 122 rather than the fluid passageway 112 within the needle hub 102. Of course, it is to be understood and appreciated that for at least one embodiment, there are a plurality of distinct porous plugs 132, which may be disposed in or about the sidewall 118 as well as in the top 134 as shown in FIG. 1C, and/or at or as a component of the inlet 106, and/or disposed in the flexible tubing 122.

The porous plug 132 is structured and arranged to distinguish gas from liquid. More specifically, the porous plug 132 is structured and arranged such that gas is permitted to pass from the fluid passageway through the porous plug 132 and of the at least one vent 130, but liquid is not. As used herein the terms “air” and “gas” are essentially interchangeable. When open and exposed to the natural environment, air will be permitted to enter the unsealed VNH 100. During packaging and sterilization of the VNH 100, in some embodiments, the assembled VNH 100 may be treated with an inert gas for enhanced sterilization. As such, “gas” is used herein in addition to “air” so as to convey an understanding that the present invention relates to embodiments where the VNH 100 may initially contain either “air” or some other “gas”.

For at least one embodiment, the porous plug 132 is formed at least in part of a hydrophobic material. For yet another embodiment, the porous plug 132 is formed of a plurality of micro capillary tubes 140 within a structure that would provide high surface tension to prevent liquid to pass, but still allow the passage of gas. A conceptualization of a plurality of micro capillary tubes 140 is shown in the enlarged section 142 of porous plug 132 in FIG. 1C.

Moreover, it is understood and appreciated that the porous plug 132 may be understood and appreciated as a selective vent structure, which is provided by one or more selective materials, or provided by one or more structures, or combinations of materials and structures so as to permit the passage of gas therethrough, but not liquid. More specifically, the term porous plug 132 as used herein has been adopted for convenience and ease of description, but is understood and appreciated to encompass materials and structures which in other settings might not be considered as “plug” material.

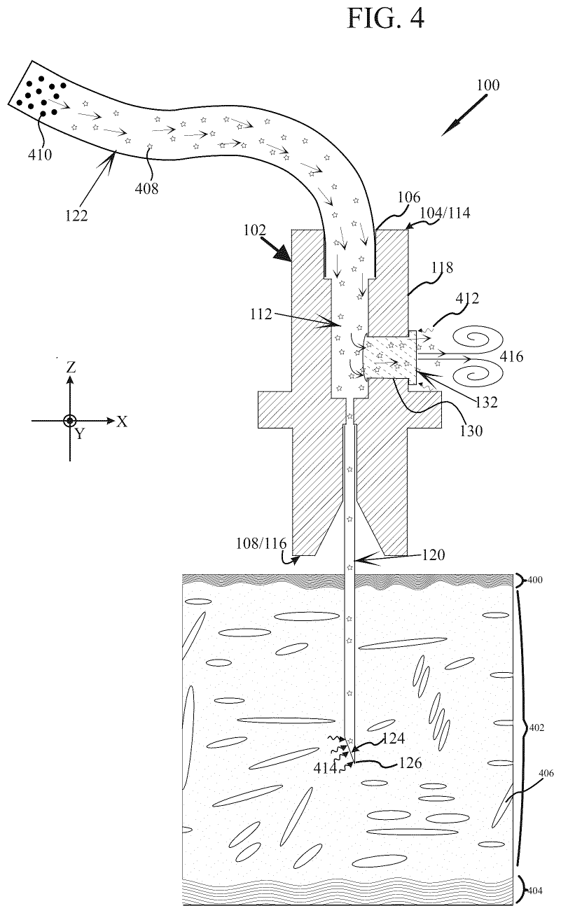

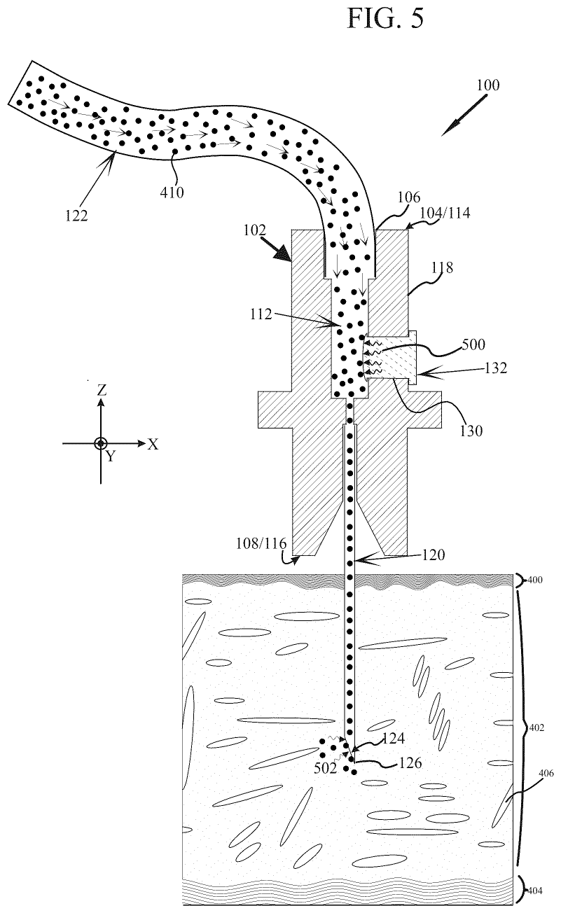

For example, in at least one embodiment, the porous plug 132 transitions to essentially a solid substance when exposed to a liquid. For example, for at least one embodiment, the porous plug 132 is provided at least in part by material that will swell when contacted by liquids, and as such pathways/channels/ducts or the like through the porous plug become even smaller and impassable to liquid, such that the porous plug 132 is a more “solid” structure then when it was dry. As will be more fully appreciated in the description below with respect to FIGS. 4 and 5, for at least one embodiment the porous plug 132 has a first air flow resistance that is lower than a second air flow resistance of subcutaneous tissue, and a first liquid flow resistance that is higher than a second liquid flow resistance of subcutaneous tissue.

In other words, for at least one embodiment, the porous plug 132 is structured and arranged such that air or other gas will more easily pass through the porous plug 132 and out of the system rather than into the subcutaneous tissues of the patient, but liquid will more easily pass into the subcutaneous tissues of the patient then through the porous plug 132 and out of the system. In truly simple terms- the porous plug 132 advantageously permits the VNH 100 to be self-priming.

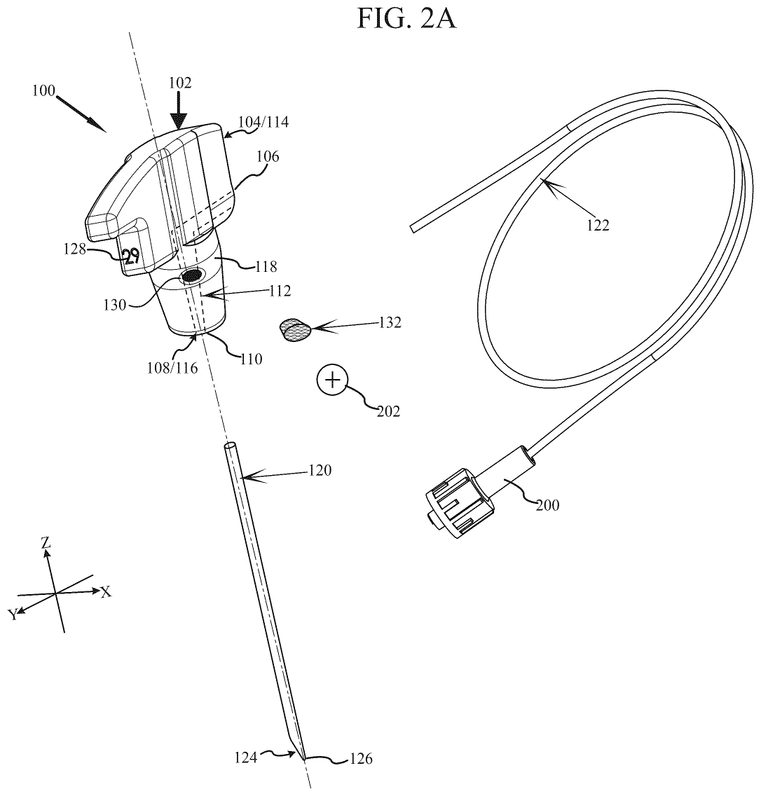

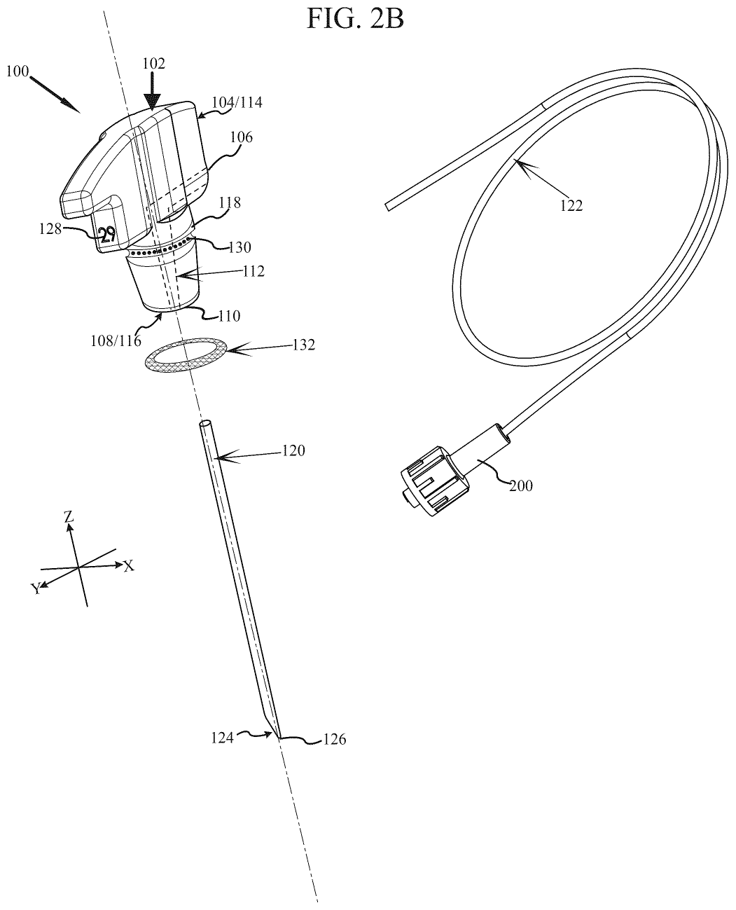

FIG. 2A provides an exploded view of the VNH 100 shown in FIG. 1A, and FIG. 2B provides an exploded view of the VNH 100 shown in FIG. 1B. In FIG. 2A, the vent 130 now exposed may be more fully appreciated, as well as the now removed and exposed porous plug 132. In FIG. 2B, the plurality of vents 130 disposed in the needle hub 102 may be appreciated with the annular ring porous plug 132 now removed.

In FIG. 2A there is also shown an optional one-way valve 202 that may be disposed over the outer surface of the porous plug 132. For at least one embodiment, the one-way valve may be a flexible membrane with one or more angled slits therein structured and arranged to open for gas passage in a first direction, but to close and seal against gas passage in a second direction. A one-way valve may also be provided for the annular ring embodiment of the porous plug 132 shown in FIGS. 1B and 2B, but for ease of illustration has not been shown.

The exploded views of FIGS. 2A & 2B also provide a conceptual representation of the flexible tubing 122, which as noted above for at least one embodiment is flow control tubing. As is also shown, for at least one embodiment the tubing 122 is coupled to a luer 200 which may be connected to a liquid reservoir or other liquid source. For at least one embodiment, the luer 200 is a flared luer as set forth in U.S. Pat. No. 10,500,389 entitled, SYSTEM AND METHOD FOR FLARED LUER CONNECTOR FOR MEDICAL TUBING, incorporated herein by reference. For yet another embodiment, the lure 200 is a tapered luer as set forth in U.S. Provisional Application 63/616,368 entitled, SYSTEM AND METHOD FOR A TAPERED LURE CONNECTOR FOR MEDICAL TUBING, incorporated herein by reference.

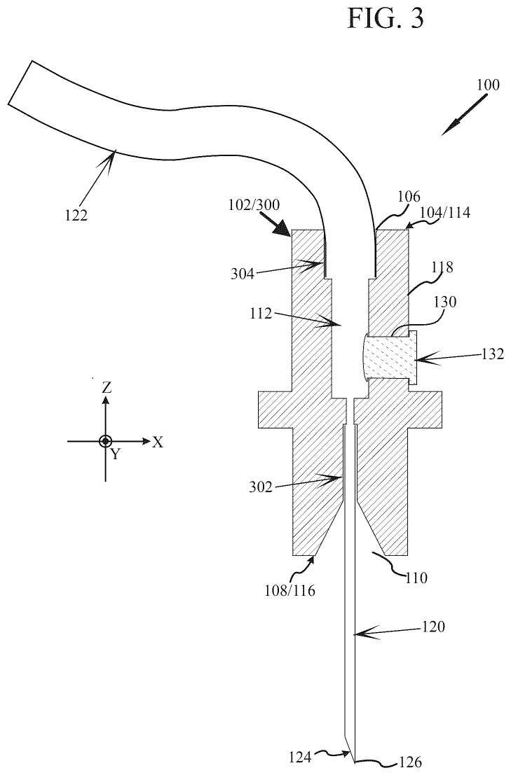

FIG. 3 provides a simplified cut through view of an exemplary embodiment of VNH 100. For ease of illustration and discussion, in this particular embedment, the inlet side 104 and the outlet side 108, and more specifically the inlet 106 and the outlet 110 have been shown to be directly opposite one another. The fluid passageway 112 between the inlet 106 and the outlet 110 may also be more fully appreciated, as well as the porous plug 132 disposed in the vent 130, such that the porous plug 132 is in fluid contact with the fluid passageway 112, but does not block or otherwise significantly impede flow as between the inlet 106 and the outlet 110.

With respect to FIG. 3, it may also be appreciated that an alternative VNH 100 is depicted, wherein the needle hub 102/300 is formed with a needle receiving section 302 such that the infusion needle 120 may be press fit, and or glued into place in a reliable and consistent fabrication process ensuring a consistent and pre-determined length of the infusion needle beyond the outlet side 108. Likewise, the needle hub 102 is formed with a flexible tubing receiving section 304 such that the end of the flexible tubing 122 may be press fit and or glued into place in a reliable and consist fabrication process.

FIGS. 4 and 5 illustrate the advantageous self-priming nature of the VNH 100, with FIG. 4 first illustrating the advantageous ability of the VNH 100 to vent air/gas from the system and FIG. 5 then showing the delivery of liquid into the patient without operator adjustment to the VNH 100

In FIG. 4, the infusion needle 120 of VNH 100 has been disposed into a patient. For ease of illustration and discussion, an enlarged portion of the patient has been rendered, showing the skin 400, and subcutaneous tissue 402, deeper muscle tissue 404 and structures such as connective tissue 406. As may be easily appreciated, the opening 124 proximate to the distal tip 126 is now disposed in the intended subcutaneous tissues 400. Moreover, the opening 124 is no longer exposed to the outside environment, e.g., air, as it is surrounded by the patient's tissues and associated liquids. It will also be appreciated that the vent 130, and more specifically the porous plug 132 is outside of the patient and therefore exposed to air or at least the gas environment in which the infusion process is being performed.

As is shown in FIG. 4, air/gas (shown as starts 408) is moving through the flexible tubing 122 as a result of liquid (shown as dots 410) being driven into the flexible tubing 122. As noted above, it may now be further appreciated that the porous plug 132 has a first air flow resistance (shown as week arrows 412) that is lower than a second air flow resistance (shown as strong arrows 414) of the subcutaneous tissues-which results in the air/gas 402 venting 416 from the VNH 100 through the porous plug 132 rather than being forced into the subcutaneous tissues 400.

In FIG. 5, the air/gas 408 within the infusion system as shown in FIG. 4 has been substantially vented through the porous plug 132 to the point that no air/gas 408 is shown in FIG. 5 and liquid 410 has now arrived from the flexible tubing 122 into the needle hub 102, and more specifically the liquid 404 has now made its way through the fluid passageway 112 and made contact with the porous plug 132. For at least one embodiment, the porous plug 132 has a first liquid flow resistance (shown as strong arrows 500) that is higher than the second liquid flow resistance (shown as week arrows 502) or the subcutaneous tissues 400. As such, and as conceptually illustrated in FIG. 5, the liquid therefore cannot pass through the porous plug 132 and is instead directed down through the infusion needle 120 and into the subcutaneous tissues 400 of the patient.

Moreover, as conceptually illustrated by FIGS. 4 and 5, the porous plug 132 is structured and arranged to distinguish between air/gas 408 and liquid 410, air/gas 408 permitted to pass from the fluid passageway 112 through the porous plug 132 while liquid 410 is not.

For at least one embodiment this advantageous nature of the porous plug 132 is achieved at least in part by the porous plug 132 being formed at least in part from a hydrophobic material. For at least one alternative embodiment the porous plug 132 may be formed at least in part from a transitional material, structured and arranged to transition from a porous state to a solid state. More specifically, this transitional material initially in a dry state provides a plurality of channels or pathways therethrough making it porous—these channels or pathways allowing gas to pass therethrough while the transitional material is in a dry state. When exposed to a liquid containing water, the transitional material swells or otherwise transitions to a solid state and in so doing closes the channels or pathways preventing further venting of gas or liquid.

As such, it may be appreciated that the VNH 100 may be disposed into the tissues of the patient without pre-priming of the system, and therefore VNH 100 advantageously avoids issues with removing air from the infusion set, wasted liquid, potential mess, discomfort and/or paid caused by the dispersion of liquid upon the patient or about the infusion needle, and possible issues of the liquid frustrating the process of temporarily adhering the VNH 100 to the patient for the duration of the infusion treatment.

For at least one embodiment, the porous plug 132 may further include a water activated color agent. As such the porous plug 132 can and will provide a visual indication to the user and/or patient that liquid has reached the needle hub 102, as the liquid contacting the porous plug 132 causes the porous plug 132 to transition from a first color 144 to a second color 146, as shown in FIG. 1C. In other words, for at least one embodiment VNH 100, and more specifically the porous plug 132, advantageously provides visual indication that the infusion set has been primed.

It will be understood and appreciated that embodiments of VNH 100 may be advantageously incorporated in various subcutaneous infusion systems. KORU Medical Systems, Inc. of Mahwah, New Jersey, is and has been a pioneer in needle set technology and flow rate control by means of specifically engineered flow control tubing. Indeed, KORU has realized that different flow rates may be provided by working with different flow combinations of flow control tubing, such as those systems and methods set forth in U.S. Pat. No. 10,420,886 entitled MULTI-FLOW UNIVERSAL TUBING SET, incorporated herein by reference, and U.S. Pat. No. 10,709,839 entitled PRECISION VARIABLE FLOW RATE INFUSION SYSTEM AND METHOD, incorporated herein by reference.

Further, KORU has developed advantageous infusion systems permitting high flow at low pressure as set forth in U.S. application Ser. No. 17/729,914 published as US 2022/0265923 entitled HIGH FLOW AT LOW PRESSURE INFUSION SYSTEM, incorporated herein by reference. Further still, for at least one embodiment the needle hub 100 is the snap in needle structure shown and described as element 162 in U.S. patent application Ser. No. 18/216,342 entitled SYSTEM AND METHOD FOR BUTTERFLY NEEDLE ASSEMBLY, incorporated herein by reference. Moreover, for at least one embodiment, the needle hub 100 is disposed in a butterfly needle assembly.

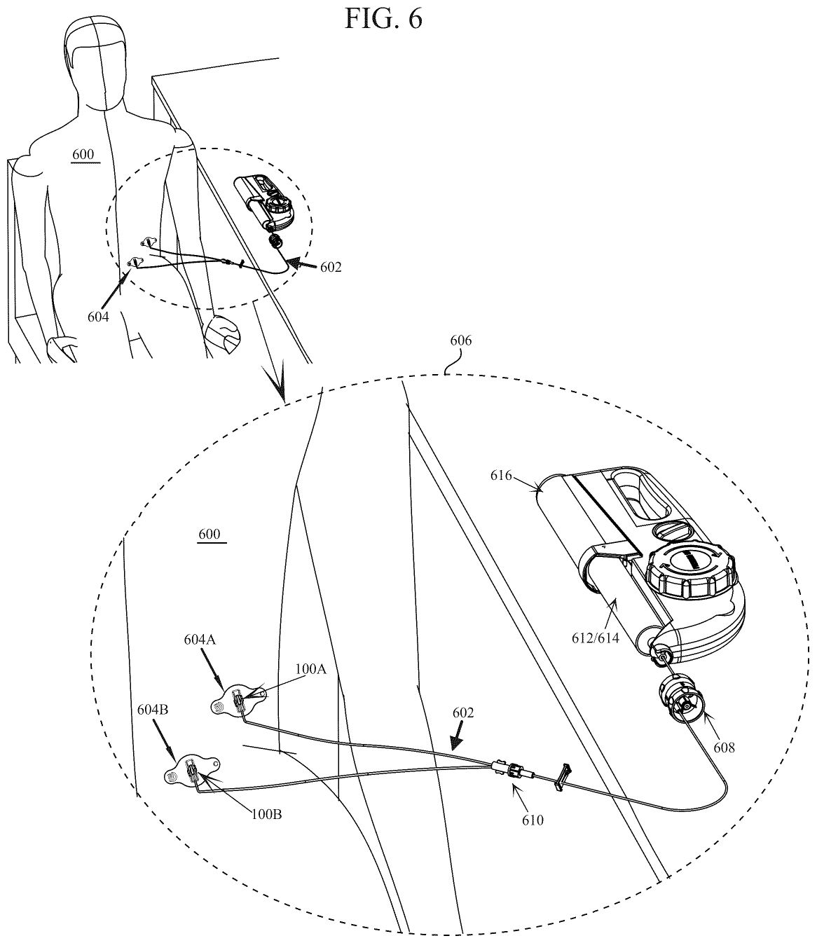

FIG. 6 presents a conceptualized infusion session for a patient 600. More specifically, a needle set 602 incorporating two butterfly needles 604A and 604B, each incorporating a VNH 100 has been disposed into a patient 600 in the lower torso area. As butterfly needles 604A and 604B have the advantageous adhesive on the generally flat bottom, no further tape is required.

As may be more fully appreciated in enlarged oval section 606, the needle set 602 is connected to a flow controller 608—this connection may be through a luer 610, or optionally the flow controller 608 may be provided as an established and inseparable element of the needle set 602. Needle set 602 is in turn connected to a liquid source or reservoir 612, such as a syringe 614.

For at least one embodiment, it is understood and appreciated that the needle set 602 is advantageously structured and arranged for use with a constant pressure pump 616, such as the Freedom60® Syringe Infusion Pump or the FreedomEdge Syringe Infusion Pump as provided by KORU Medical Systems, Inc. of Mahwah, New Jersey. Constant pressure systems, such as constant pressure pump 616, when combined with butterfly needles incorporating VNH 100, and more specifically needle sets such as needle set 602 may be highly advantageous in being self-priming to eliminate issues noted above as well as avoiding the discomfort and possible complications of introducing gas into the subcutaneous tissues.

Although the above description and illustrations have been presented with respect to subcutaneous infusion, it will be understood and appreciated that the advantageous self-priming nature of the VNH 100 to remove air from the system is advantageously adaptable to IV infusion systems and methods as well.

Moreover, with respect to the above description, at least one embodiment of VNH 100 may be summarized as a needle hub 102 having an inlet side 104 and an outlet side 108 and a fluid passageway 112 therebetween; an infusion needle in fluid contact with the fluid passageway 112 and extending from the outlet side 108; a flexible tubing 122 joined to the inlet side 104 in fluid contact with the fluid passageway 112; at least one vent 130 disposed in a sidewall 118 of the needle hub 102, the vent 130 in fluid contact with the fluid passageway 112; and a porous plug 132 disposed within each at least one vent 130 adjacent to the fluid passageway, the porous plug 132 structured and arranged to distinguish gas 402 from liquid 404, gas 402 permitted to pass from the fluid passageway 112 through the porous plug 132 and out the at least one vent 130.

Further, an embodiment of VNH 100 may also be summarized as a needle hub 102 having a tubing 122 inlet at a first location and a needle extending from a second location, and at least one sidewall; a fluid passageway between the tubing 122 inlet and the needle; at least one vent 130 in the needle hub 102 between the first location and the second location, the at least one vent 130 in fluid communication with the fluid passageway; and a porous plug 132 disposed within each at least one vent 130 adjacent to the fluid passageway; wherein the porous plug 132 has a first air flow resistance lower than a second air flow resistance of subcutaneous tissue, and a first liquid 404 flow resistance higher than second liquid 404 flow resistance of subcutaneous tissue.

Still yet another embodiment of VNH 100 may be summarized as a needle hub 102 having a first end and a second end and a fluid passageway 112 therebetween, the first end joined with a flexible tubing 122 line and the second end joined with a needle having a first length; at least one vent 130 in the needle hub 102 between the first location and the second location, the at least one vent 130 in fluid communication with the fluid passageway; and a porous plug 132 disposed within each at least one vent 130 adjacent to the fluid passageway, the porous plug 132 structured and arranged to permit gas 402 to pass from the fluid passageway 112 through the porous plug 132 and out the at least one vent 130.

Having described embodiments of the VNH 100, other embodiments relating to at least one method of providing a VNH 100, as well as at least one method of providing infusion therapy into subcutaneous tissues with a VNH 100 will now be discussed. It will be appreciated that the described methods need not be performed in the order in which they are herein described, but that these descriptions are merely exemplary of methods to provide and use a VNH 100.

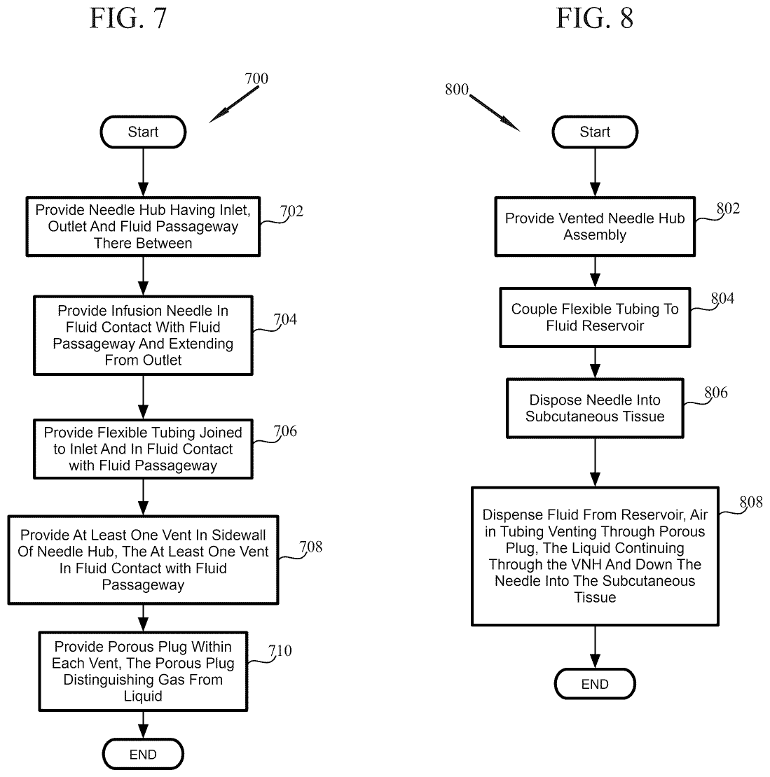

Turning to FIG. 7, presented is a flow diagram for at least one method 700 of providing a VNH 100. In general, method 700 commences with providing a needle hub 102 having an inlet 106 and an outlet 110 and a fluid passageway 112 therebetween, block 702. As discussed above, for at least one embodiment, the inlet 106 is provided by an inlet side 104 and the outlet is provided by an outlet side 108.

An infusion needle 120 is also provided in fluid contact with the fluid passageway 112 and extending from the outlet side, block 704. A flexible tubing 122 is also provided and joined to the inlet 106 of the inlet side 106 in fluid contact with the fluid passageway 112, block 706. At least one vent 130 is also provided in the sidewall of the needle hub, the vent 130 in fluid communication with the fluid passageway 112, block 708. Of course, it will be understood and appreciated that for at least one embodiment, the at least one vent may be established during the process of forming the needle hub 102 such as by 3D printing, stamping, casting or such other fabrication process.

Method 700 concludes with providing a porous plug 132 in each of the provided vents 130, the porous plug 132 structured and arranged to distinguish gas from liquid, gas permitted to pass from the passageway 112 through the porous plug 132 and out the at least one vent 130, block 710.

Moreover, method 700 may be summarized as providing a needle hub 102 having an inlet side 104 and an outlet side 108 and a fluid passageway 112 therebetween; providing an infusion needle in fluid contact with the fluid passageway 112 and extending from the outlet side 108; providing a flexible tubing 122 joined to the inlet side 104 in fluid contact with the fluid passageway 112; providing at least one vent 130 disposed in a sidewall 118 of the needle hub 102, the vent 130 in fluid contact with the fluid passageway 112; providing a porous plug 132 disposed within each at least one vent 130 adjacent to the fluid passageway, the porous plug 132 structured and arranged to distinguish gas 402 from liquid 404, gas 402 permitted to pass from the fluid passageway 112 through the porous plug 132 and out the at least one vent 130.

Turning to FIG. 8, presented is a flow diagram for at least one method 800 of providing infusion therapy with a VNH 100. In general, method 800 commenced with providing a VNH 100 in accordance with at least one of the embodiments discussed and described above, block 802.

The flexible tubing is coupled to a liquid reservoir, such as liquid reservoir 612 shown in FIG. 6, block 804. It will be understood and appreciated that in the initial starting state, there is some quantity of air or gas in the VNH 100. Method 800 continues with disposing the needle into the subcutaneous tissue of a patient, block 806. Now, with the needle properly disposed into the subcutaneous tissues of the patient, the infusion method 800 continues with the dispensing of the liquid from the reservoir into the flexible tubing 122 which in turn drives the air/gas within the tubing towards the needle hub 102, block 808.

As described above, as the porous plug 132 is structured and arranged to distinguish between gas and liquid, this air/gas advantageously vents through the porous plug 132. As the liquid arrives within the needle hub 102, the porous plug 132 does not permit the liquid to pass through and out the vent 130, and the liquid is therefore further directed through the infusion needle 120 and into the subcutaneous tissue, block 808.

Moreover, method 800 may be summarized as providing a needle hub 102 having: an inlet side 104 and an outlet side 108 and a fluid passageway 112 therebetween; an infusion needle in fluid contact with the fluid passageway 112 and extending from the outlet side 108; a flexible tubing 122 joined to the inlet side 104 in fluid contact with the fluid passageway 112; at least one vent 130 disposed in a sidewall 118 of the needle hub 102, the vent 130 in fluid contact with the fluid passageway 112; a porous plug 132 disposed within each at least one vent 130 adjacent to the fluid passageway, the porous plug 132 structured and arranged to distinguish gas 402 from liquid 404, gas 402 permitted to pass from the fluid passageway 112 through the porous plug 132 and out the at least one vent 130; coupling the flexible tubing 122 to a fluid reservoir, the flexible tubing 122 containing a quantity of air; disposing the needle into the subcutaneous tissue of a patient; dispensing fluid from the reservoir into the flexible tubing 122, the fluid propagating the air towards the needle hub 102, the air venting through the porous plug 132, the fluid reaching the porous plug 132 and continuing down through the needle into the subcutaneous tissues.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A vented needle hub assembly for infusion of a liquid into subcutaneous tissue, comprising:
a needle hub having an inlet side and an outlet side and a fluid passageway therebetween;
an infusion needle in fluid contact with the fluid passageway and extending from the outlet side;
a flexible tubing joined to the inlet side in fluid contact with the fluid passageway;
at least one vent disposed in a sidewall of the needle hub, the vent in fluid contact with the fluid passageway; and
a porous plug disposed within each at least one vent adjacent to the fluid passageway, the porous plug structured and arranged to distinguish gas from liquid, gas permitted to pass from the fluid passageway through the porous plug and out the at least one vent.

2. The vented needle hub assembly of claim 1, wherein the porous plug is formed at least in part of hydrophobic material.

3. The vented needle hub assembly of claim 1, wherein the porous plug transitions to essentially a solid substance when exposed to a liquid.

4. The vented needle hub assembly of claim 1, wherein the porous is provided at least in part by a plurality of micro capillary tubes structured and arranged to provide a high surface tension to prevent the passage of liquid while permitting the passage of gas.

5. The vented needle hub assembly of claim 1, wherein the porous plug transitions from a first color to a second color for visual indication that the vented needle hub assembly has been primed.

6. The vented needle hub assembly of claim 1, wherein the porous plug has a first air flow resistance lower than a second air flow resistance of subcutaneous tissue, and a first liquid flow resistance higher than second liquid flow resistance of subcutaneous tissue.

7. The vented needle hub assembly of claim 1, wherein the inlet side and the outlet side are parallel to each other.

8. The vented needle hub assembly of claim 1, wherein the inlet side and the outlet side are at normal to each other.

9. The vented needle hub assembly of claim 1, wherein the sidewall of the needle hub is the top.

10. The vented needle hub assembly of claim 1, wherein the needle is a straight needle.

11. The vented needle hub assembly of claim 1, wherein a one-way valve is disposed upon the exterior of the vent, the one-way valve structured and arranged to permit gas to exit from the needle hub.

12. The vented needle hub assembly of claim 1, wherein the vented needle hub assembly is disposed in a butterfly needle assembly.

13. A vented needle hub assembly for infusion of a liquid into subcutaneous tissue, comprising:
a needle hub having a tubing inlet at a first location and a needle extending from a second location, and at least one sidewall;
a fluid passageway between the tubing inlet and the needle;
at least one vent in the needle hub between the first location and the second location, the at least one vent in fluid communication with the fluid passageway; and
a porous plug disposed within each at least one vent adjacent to the fluid passageway;
wherein the porous plug has a first air flow resistance lower than a second air flow resistance of subcutaneous tissue, and a first liquid flow resistance higher than second liquid flow resistance of subcutaneous tissue.

14. The vented needle hub assembly of claim 13, wherein the porous plug is formed at least in part of hydrophobic material.

15. The vented needle hub assembly of claim 13, wherein the porous plug transitions to essentially a solid substance when exposed to a liquid.

16. The vented needle hub assembly of claim 13, wherein the porous is provided at least in part by a plurality of micro capillary tubes structured and arranged to provide a high surface tension to prevent the passage of liquid while permitting the passage of gas.

17. The vented needle hub assembly of claim 13, wherein the porous plug transitions from a first color to a second color for visual indication that the vented needle hub assembly has been primed.

18. The vented needle hub assembly of claim 13, wherein the first location and the second location are opposite each other.

19. The vented needle hub assembly of claim 13, wherein the first location and the second location are normal to each other.

20. The vented needle hub assembly of claim 13, wherein a one-way valve is disposed upon the exterior of the vent, the one-way valve structured and arranged to permit gas to exit from the needle hub.

21. The vented needle hub assembly of claim 13, wherein the sidewall of the needle hub is the top.

22. The vented needle hub assembly of claim 13, wherein the needle is a straight needle.

23. The vented needle hub assembly of claim 13, wherein the vented needle hub assembly is disposed in a butterfly needle assembly.

24. A vented needle hub assembly for infusion of a liquid into subcutaneous tissue, comprising:
a needle hub having a first end and a second end and a fluid passageway therebetween, the first end joined with a flexible tubing line and the second end joined with a needle having a first length;
at least one vent in the needle hub between the first location and the second location, the at least one vent in fluid communication with the fluid passageway;
a porous plug disposed within each at least one vent adjacent to the fluid passageway, the porous plug structured and arranged to permit gas to pass from the fluid passageway through the porous plug and out the at least one vent.

25. The vented needle hub assembly of claim 24, wherein the porous plug is formed at least in part of hydrophobic material.

26. The vented needle hub assembly of claim 24, wherein the porous plug transitions to essentially a solid substance when exposed to a liquid.

27. The vented needle hub assembly of claim 24, wherein the porous is provided at least in part by a plurality of micro capillary tubes structured and arranged to provide a high surface tension to prevent the passage of liquid while permitting the passage of gas.

28. The vented needle hub assembly of claim 24, wherein the porous plug transitions from a first color to a second color for visual indication that the vented needle hub assembly has been primed.

29. The vented needle hub assembly of claim 24, wherein the porous plug has a first air flow resistance lower than a second air flow resistance of subcutaneous tissue, and a first liquid flow resistance higher than second liquid flow resistance of subcutaneous tissue.

30. The vented needle hub assembly of claim 24, wherein the first end and the second end are parallel to each other.

31. The vented needle hub assembly of claim 24, wherein the first end and the second end are at normal to each other.

32. The vented needle hub assembly of claim 13, wherein the sidewall of the needle hub is the top.

33. The vented needle hub assembly of claim 24, wherein the needle is a straight needle.

34. The vented needle hub assembly of claim 24, wherein a one-way valve is disposed upon the exterior of the vent, the one-way valve structured and arranged to permit gas to exit from the needle hub.

35. The vented needle hub assembly of claim 24, wherein the vented needle hub assembly is disposed in a butterfly needle assembly.

36. A method of providing a vented needle hub assembly for infusion of a liquid into subcutaneous tissue, comprising:
providing a needle hub having an inlet side and an outlet side and a fluid passageway therebetween;
providing an infusion needle in fluid contact with the fluid passageway and extending from the outlet side;
providing a flexible tubing joined to the inlet side in fluid contact with the fluid passageway;
providing at least one vent disposed in a sidewall of the needle hub, the vent in fluid contact with the fluid passageway;
providing a porous plug disposed within each at least one vent adjacent to the fluid passageway, the porous plug structured and arranged to distinguish gas from liquid, gas permitted to pass from the fluid passageway through the porous plug and out the at least one vent.

37. The method of claim 36, wherein the porous plug is formed at least in part of hydrophobic material.

38. The method of claim 36, wherein the porous plug transitions to essentially a solid substance when exposed to a liquid.

39. The method of claim 36, wherein the porous is provided at least in part by a plurality of micro capillary tubes structured and arranged to provide a high surface tension to prevent the passage of liquid while permitting the passage of gas.

40. The method of claim 36, wherein the porous plug transitions from a first color to a second color for visual indication that the vented needle hub assembly has been primed.

41. The method of claim 36, wherein the porous plug has a first air flow resistance lower than a second air flow resistance of subcutaneous tissue, and a first liquid flow resistance higher than second liquid flow resistance of subcutaneous tissue.

42. The method of claim 36, wherein the inlet side and the outlet side are parallel to each other.

43. The method of claim 36, wherein the inlet side and the outlet side are at normal to each other.

44. A method of providing infusion therapy into subcutaneous tissue with a vented needle hub assembly, comprising:
providing a needle hub having:
an inlet side and an outlet side and a fluid passageway therebetween;
an infusion needle in fluid contact with the fluid passageway and extending from the outlet side;
a flexible tubing joined to the inlet side in fluid contact with the fluid passageway;
at least one vent disposed in a sidewall of the needle hub, the vent in fluid contact with the fluid passageway;
a porous plug disposed within each at least one vent adjacent to the fluid passageway, the porous plug structured and arranged to distinguish gas from liquid, gas permitted to pass from the fluid passageway through the porous plug and out the at least one vent;
coupling the flexible tubing to a liquid reservoir, the flexible tubing containing a quantity of air;
disposing the needle into the subcutaneous tissue of a patient;
dispensing liquid from the reservoir into the flexible tubing, the liquid propagating the air towards the needle hub, the air venting through the porous plug, the liquid reaching the porous plug and continuing down through the needle into the subcutaneous tissues.

45. The method of providing infusion therapy of claim 44, wherein the liquid is dispensed from the reservoir by a constant pressure pump.

46. The method of providing infusion therapy of claim 44, wherein the method permits infusion without purging.

47. The method of providing infusion therapy of claim 44, wherein the porous plug is formed at least in part of hydrophobic material.

48. The method of providing infusion therapy of claim 44, wherein the porous plug transitions to essentially a solid substance when exposed to a liquid.

49. The method of providing infusion therapy of claim 44, wherein the porous is provided at least in part by a plurality of micro capillary tubes structured and arranged to provide a high surface tension to prevent the passage of liquid while permitting the passage of gas.

50. The method of providing infusion therapy of claim 44, wherein the porous plug transitions from a first color to a second color for visual indication that the vented needle hub assembly has been primed.

51. The method of providing infusion therapy of claim 44, wherein the porous plug has a first air flow resistance lower than a second air flow resistance of subcutaneous tissue, and a first liquid flow resistance higher than second liquid flow resistance of subcutaneous tissue.
